(19) United States
(12) Patent Application Publication
(10) Pub. No.: US 2018/0078677 A1
(43) Pub. Date: Mar. 22, 2018

(54) COMPOSITION FOR THREE-DIMENSIONAL PRINTING, METHOD FOR PREPARING SAME, AND METHOD FOR MANUFACTURING THREE-DIMENSIONAL STRUCTURE USING SAME

(71) Applicants: POSTECH ACADEMY-INDUSTRY FOUNDATION, Pohang-si, Gyeongsangbuk-do (KR); T&R BIOFAB CO., LTD., Siheung-si, Gyeonggi-do (KR)

(72) Inventors: Dong-Woo CHO, Seoul (KR); Jin-Ah JANG, Seoul (KR)

(73) Assignees: POSTECH ACADEMY-INDUSTRY FOUNDATION, Pohang-si, Gyeongsangbuk-do (KR); T&R BIOFAB CO., LTD., Siheung-si, Gyeonggi-do (KR)

(21) Appl. No.: 15/561,350

(22) PCT Filed: Feb. 25, 2016
(86) PCT No.: PCT/KR2016/001839

§ 371 Date: Sep. 25, 2017

(30) Foreign Application Priority Data
Mar. 26, 2015 (KR) 10-2015-0042412

(51) Int. Cl. A61L 27/36; A61L 27/22; A61L 27/26; A61L 27/18; B33Y 10/00; B33Y 70/00

(52) U.S. Cl. CPC A61L 27/3687; A61L 27/3633; A61L 27/227; B33Y 70/00; A61L 27/18; B33Y 10/00; A61L 27/26

(57) ABSTRACT

Provided is a three-dimensional printing composition including decellularized extracellular matrix; and riboflavin as a crosslinking agent. A three-dimensional structure having high mechanical strength can be prepared by performing a printing process using the three-dimensional printing composition according to the present invention and a layer-by-layer process through crosslinking under UVA light to prepare a three-dimensional structure configuration; and then performing thermal gelation of the three-dimensional structure configuration. Further provided is a method for preparing said three-dimensional printing composition and a method for preparing a three-dimensional structure using said three-dimensional printing composition.

### TECHNICAL FIELD

The present invention relates to a three-dimensional printing composition. And also, the present invention relates to a method for preparing said three-dimensional printing composition and a method for preparing a three-dimensional structure using said three-dimensional printing composition.

### BACKGROUND ART

Three-dimensional printing refers to fabricating a complicated skeletal structure through converting the configuration information derived from medical data of the tissues or organs having complicated configurations to the G-codes and then performing a layer-by-layer process using the same. Such a three-dimensional printing is also referred to as ‘three-dimensional bioprinting (3D bioprinting)’. For example, ‘the multi-head tissue/organ printing system’, which is one of the representative three-dimensional printing techniques, consists of two pneumatic syringes for injecting materials by air pressure and two piston syringes for injecting materials in a nano-liter unit using a step motor, thereby being capable of utilizing various materials at the same time. Typically, thermoplastic biocompatible polymers, such as PLA (polylactic Acid), PGA (poly-glycolic acid), PLGA (poly-lactic-co-glycolid acid), PCL (polycaprolactone), or a mixture thereof, are loaded into the pneumatic syringes, so as to prepare a three-dimensional structure. In addition, hydrogels, including collagen, hyaluronic acid, gelatin, alginate, chitosan or fibrin and the like, are loaded into the piston syringes, so as to prepare a three-dimensional structure.

A crucial aspect of bioprinting is that the printing process should be cytocompatible, as it requires the dispensing of cell-containing media. This restriction reduces the choice of materials because of the necessity to operate in an aqueous or aqueous gel environment. Therefore, hydrogels using the materials such as gelatin, gelatin/chitosan, gelatin/alginate, gelatin/fibronectin, Lutrol F127/alginate, alginate and the like are being used for preparing diverse tissues ranging from liver to bone. Said hydrogels or mixtures of hydrogel and cells used for bioprinting are also referred to as ‘bioink’.

Normally, cells remain located specifically in their original deposited position during the whole culture period, as they are unable to adhere or degrade the surrounding alginate gel matrix (Fedorovich, N. E. et al. Tissue Eng. 13, 1905-1925 (2007)). Thus, although there were some successful reports about bioprinting of cell-printed structure, minimal cells-material interactions and inferior tissue formation are the foremost concerns. Actually, these materials cannot represent the complexity of natural extracellular matrices (ECMs) and thus are inadequate to recreate a microenvironment with cell-cell connections and three-dimensional (3D) cellular organization that are typical of living tissues. Consequently, the cells in those hydrogels cannot exhibit intrinsic morphologies and functions of living tissues in vivo. It is thus ideal if cells are provided the natural microenvironment similar to their parent tissue. Decellularized extracellular matrix (dECM) is the best choice for doing so, as no natural or man-made material can recapitulate all the features of natural extracellular matrix. Moreover, ECM of each tissue is unique in terms of composition and topology, which is generated through dynamic and reciprocal interactions between the resident cells and microenvironment. Recent studies of cells and ECM isolated from tissues and organs highlight the necessity of tissue specificity for preserving selected cell functions and phenotype (Sellaro, T. L. et al. Tissue Eng. Part A 16, 1075-1082 (2010); Petersen, T. H. et al. Science 329, 538-541 (2010); Uygun, B. E. et al. Nat. Med. 16, 814-821 (2010); Ott, H. C. et al. Nat. Med. 16, 927-933 (2010); Flynn, L. E. Biomaterials 31, 4715-4724 (2010)). The dECM materials are harvested and typically processed as two-dimensional (2D) scaffolds from various tissues, including skin, small intestinal submucosa, where at the initial stages the infiltrating or seeded cell populations depend on diffusion of oxygen and nutrient for their survival until a supporting vascular network develops. However, printing tissue analogue structures requires a fabrication approach to devise a highly open porous 3D structure to allow the flow of nutrients. The present inventors have developed a three-dimensional printing method for printing cell-laden constructs with the use of dECM bioink capable of providing an optimized microenvironment conducive to the growth of three-dimensional structured tissue. The cell-laden constructs are able to reconstitute the intrinsic cellular morphologies and functions (Falguni Pati, et al., Nat Commun. 5, 3935 (2014)).

Meanwhile, the structure prepared by three-dimensional printing with a dECM bioink should have a mechanical strength capable of maintaining the three-dimensional configuration. For example, in extrusion-based printing such as a multi-head tissue/organ printing system, a three-dimensional structure configuration is formed while the bioink in a pre-gel form is being maintained at a temperature below about 15° C. during the extrusion from the syringe. The resulting three-dimensional structure configuration is subject to thermal processing or post-print crosslinking, as a process for imparting an appropriate mechanical strength. The thermal processing is carried out for example through gelation in a humid incubator at about 37° C. The post-print crosslinking is carried out through crosslinking by treating the three-dimensional structure configuration with a solution of crosslinking agent such as glutaraldehyde. However, since the three-dimensional structure obtained through gelation by the thermal processing shows relatively low mechanical strength, it is difficult to manufacture the organ that requires satisfactory mechanical strength. And also, post-print crosslinking requires the use of toxic cross-linking agents, such as glutaraldehyde, which causes a safety problem. In addition, the insufficient crosslinking inside the three-dimensional structure results in the problem that non-uniformly crosslinked three-dimensional structures are obtained.

### DISCLOSURE

### Technical Problem

The present inventors carried out various studies in order to develop an improved method for preparing a three-dimensional structure having high mechanical strength by three-dimensional printing processes. The present inventors have developed a method capable of preparing a three-dimensional structure having high mechanical strength uniformly, the method of which includes performing a printing process using the three-dimensional printing composition comprising riboflavin having a high safety as a crosslinking agent and a layer-by-layer process through crosslinking under UVA light to prepare a three-dimensional structure configuration; and then performing thermal gelation of the three-dimensional structure configuration. That is, the present inventors have newly developed a crosslinking-thermal gelation method which includes the use of riboflavin.

Therefore, it is an object of the present invention to provide a three-dimensional printing composition comprising riboflavin as a crosslinking agent.

It is another object of the present invention to provide a method for preparing said three-dimensional printing composition.

It is still another object of the present invention to provide a method for preparing a three-dimensional structure using said three-dimensional printing composition.

### Technical Solution

In accordance with an aspect of the present invention, there is provided a three-dimensional printing composition comprising a decellularized extracellular matrix; and riboflavin as a crosslinking agent.

In the three-dimensional printing composition of the present invention, the decellularized extracellular matrix may be obtained by decellularization of heart tissues, cartilage tissues, bone tissues, adipose tissues, muscle tissues, skin tissue, mucosal epithelial tissues, amnion tissues, or corneal tissues which are externally discharged from the body; and may be present in an amount ranging from 1 to 4% by weight, based on the total weight of the composition. And also, the riboflavin may be present in an amount ranging from 0.001 to 0.1% by weight, based on the total weight of the composition.

The three-dimensional printing composition of the present invention may further comprise one or more acids selected from the group consisting of acetic acid and hydrochloric acid; one or more proteinases selected from the group consisting of pepsin and matrix metalloproteinase; and a pH adjusting agent. In an embodiment, the three-dimensional printing composition of the present invention may comprise 1 to 4% by weight of the decellularized extracellular matrix; 0.001 to 0.1% by weight of the riboflavin; 0.03 to 30% by weight of one or more acids selected from the group consisting of acetic acid and hydrochloric acid; 0.1 to 0.4% by weight of one or more proteinases selected from the group consisting of pepsin and matrix metalloproteinase; and a pH adjusting agent, based on the total weight of the composition. And also, in the three-dimensional printing composition of the present invention, the viscosity at 1 s⁻¹ shear rate when measured at 15° C. may range from 1 to 30 Pa·S.

In accordance with another aspect of the present invention, there is provided a method for preparing a three-dimensional printing composition comprising: (a) adding a decellularized extracellular matrix to one or more acid solutions selected from the group consisting of acetic acid and hydrochloric acid, (b) adding one or more proteinases selected from the group consisting of pepsin and matrix metalloproteinase to the solution obtained from Step (a), followed by stirring the mixture to obtain a solution, and (c) adding riboflavin and a pH adjusting agent to the solution obtained from Step (b).

In accordance with still another aspect of the present invention, there is provided a method for preparing a three-dimensional structure comprising: (i) performing a printing process using the three-dimensional printing composition and a layer-by-layer process through crosslinking under UVA light, to form a three-dimensional structure configuration; and (ii) performing thermal gelation of the three-dimensional structure configuration obtained from Step (i) at a temperature of 15° C. or more, to prepare a three-dimensional structure.

In an embodiment, the crosslinking in each layer-by-layer process may be performed for 1 to 10 minutes.

### Advantageous Effects

It has been found by the present invention that a three-dimensional structure having high mechanical strength can be prepared by performing a printing process using the three-dimensional printing composition comprising riboflavin and a layer-by-layer process through crosslinking under UVA light to prepare a three-dimensional structure configuration; and then performing thermal gelation of the three-dimensional structure configuration. That is, the present invention provides a crosslinking-thermal gelation method including the use of riboflavin, which makes it possible to prepare a three-dimensional structure having high mechanical strength uniformly. And also, the present invention includes the use of riboflavin having a high safety as a crosslinking agent, thereby being capable of avoiding the use of toxic crosslinking agents such as glutaraldehyde. Accordingly, the present invention can be usefully applied to fabricating tissue-engineering scaffolds, cell-based sensors, drug/toxicity screening models and tissue or tumour models, through three-dimensional printing.

### DESCRIPTION OF DRAWINGS

FIGS. 1a and 1 b show the optical microscopic images (FIG. 1a) and histological images (FIG. 1b) of the decellularized extracellular matrix (hdECM) derived from heart tissues.

FIG. 2 shows the configuration of the three-dimensional structure prepared according to the present invention, with using a PCL framework.

FIG. 3 shows the configuration of the three-dimensional structure prepared according to the present invention, without using a PCL framework.

### BEST MODE

The present invention provides a three-dimensional printing composition comprising a decellularized extracellular matrix; and riboflavin as a crosslinking agent.

The decellularized extracellular matrix may be obtained by decellularization of tissues discharged from mammals such as human, pig, cow, rabbit, dog, goat, sheep, chicken, horse and the like. The tissues are not particularly limited, and for example include heart tissues, cartilage tissues, bone tissues, adipose tissues, muscle tissues, skin tissue, mucosal epithelial tissues, amnion tissues, or corneal tissues, preferably heart tissues, cartilage tissues, or bone tissue, more preferably heart tissues, cartilage tissues, or bone tissues derived from pigs. The decellularization may be performed according to or with minor modifications to known methods disclosed in for example Ott, H. C. et al. Nat. Med. 14, 213-221 (2008), Yang, Z. et al. Tissue Eng. Part C Methods 16, 865-876 (2010), and the like. Preferably, the decellularization may be carried out according to the decellularization method previously reported by the present inventors, i.e., according to the decellularization method disclosed in Falguni Pati, et al., Nat Commun. 5, 3935 (2014). The obtained decellularized extracellular matrix is typically stored in a lyophilized powder form. The amount of the decellularized extracellular matrix is not particularly limited. For example, the decellularized extracellular matrix may be used in an amount ranging from 0.001 to 0.1% by weight, preferably from 2 to 3% by weigh, based on the total weight of the composition.

It has been found by the present invention that a three-dimensional structure having high mechanical strength uniformly can be prepared by performing a printing process using the three-dimensional printing composition comprising riboflavin having a high safety and a layer-by-layer process through crosslinking under UVA light to prepare a three-dimensional structure configuration; and then performing thermal gelation of the three-dimensional structure configuration. That is, the present invention provides a crosslinking-thermal gelation method including the use of riboflavin. Said riboflavin may be used in a sufficient amount for crosslinking under UVA light. For example, the riboflavin may be used in an amount ranging from 0.001 to 0.1% by weight, preferably from 0.01 to 0.1% by weight, based on the total weight of the composition.

It is preferable that the three-dimensional printing composition of the present invention is in the form of a visco-elastic homogeneous solution having a range of pH 6.5 to 7.5, in order to provide efficient three-dimensional printing. Therefore, the three-dimensional printing composition of the present invention may further comprise one or more acids selected from the group consisting of acetic acid and hydrochloric acid; one or more proteinases selected from the group consisting of pepsin and matrix metalloproteinase; and a pH adjusting agent for controlling the pH to the range of 6.5 to 7.5 (for example, sodium hydroxide), in an aqueous medium. The acid functions to dissolve a decellularized extracellular matrix. Preferably, the acid may be acetic acid, hydrochloric acid, and the like. More preferably, the acid may be used in the form of a 0.01M-10M acetic acid solution (for example, about 0.5M acetic acid solution) or in the form of a 0.01M-10M hydrochloric acid solution. The proteinase functions to digest the telopeptide in a decellularized extracellular matrix. Preferably, the proteinase may be pepsin, matrix metalloproteinase, and the like. The amount of the proteinase depends on the amount of a decellularized extracellular matrix. For example, the proteinase may be used in a ratio of 5 to 30 mg, preferably 10 to 25 mg, with respect to 100 mg of a decellularized extracellular matrix. The pH adjusting agent functions to neutralize the acid used for dissolving a decellularized extracellular matrix. For example, sodium hydroxide as the pH adjusting agent may be used in an amount sufficient to control the pH to pH 6.5 to 7.5, preferably about pH 7.

In an embodiment, the three-dimensional printing composition of the present invention may comprise 1 to 4% by weight of the decellularized extracellular matrix; 0.001 to 0.1% by weight of the riboflavin; 0.03 to 30% by weight of one or more acids selected from the group consisting of acetic acid and hydrochloric acid; 0.1 to 0.4% by weight of one or more proteinases selected from the group consisting of pepsin and matrix metalloproteinase; and a pH adjusting agent, based on the total weight of the composition. And also, the three-dimensional printing composition of the present invention is preferably in the visco-elastic form which shows lower viscosity according to increasing the shear rate thereof. For example, in the three-dimensional printing composition of the present invention, the viscosity at 1 s⁻¹ shear rate when measured at 15° C. is preferably from 1 to 30 Pa·S. The viscosity may be adjusted by appropriately controlling the amount of aqueous medium (e.g., water, distilled water, PBS, physiological saline, etc.).

The present invention also provides a method for preparing said three-dimensional printing composition. That is, the present invention provides a method for preparing a three-dimensional printing composition comprising: (a) adding a decellularized extracellular matrix to one or more acid solutions selected from the group consisting of acetic acid and hydrochloric acid, (b) adding one or more proteinases selected from the group consisting of pepsin and matrix metalloproteinase to the solution obtained from Step (a), followed by stirring the mixture to obtain a solution, and (c) adding riboflavin and a pH adjusting agent to the solution obtained from Step (b).

In the method of the present invention, the acid, decellularized extracellular matrix, riboflavin, proteinase, and pH adjusting agent are as described above.

The acid solution of Step (a) may be e.g., a 0.01M-0.5M acetic acid solution, preferably an about 0.5M acetic acid solution. The stirring of Step (b) may be carried out until achieving complete solubilization of the decellularized extracellular matrix. The stirring of Step (b) may be carried out typically for 24 to 48 hours, but not limited thereto. Step (c) is carried out at about 15° C. or less, preferably at a low temperature ranging from about 4° C. to about 10° C., in order to avoid gelation. The resulting three-dimensional printing composition is in the form of pH-adjusted pre-gel, which is stored preferably at about 4° C.

The present invention also provides a method for preparing a three-dimensional structure comprising: (i) performing a printing process using the three-dimensional printing composition and a layer-by-layer process through crosslinking under UVA light, to form a three-dimensional structure configuration; and (ii) performing thermal gelation of the three-dimensional structure configuration obtained from Step (i) at a temperature of 15° C. or more, to prepare a three-dimensional structure.

The printing of Step (i) may be carried out by using known three-dimensional printing methods (e.g., a printing method with ‘the multi-head tissue/organ printing system’), according to the methods disclosed in Falguni Pati, et al., Nat Commun. 5, 3935 (2014) and the like. For example, the printing may be performed by using two syringes of the multi-head tissue/organ printing system. That is, a polycaprolactone (PCL) framework is loaded into the syringe, followed by heating to about 80° C. to melt the polymer. Said three-dimensional printing composition in the form of pre-gel is loaded into the other syringe, followed by maintaining the temperature at about 15° C. or less, preferably at about 4° C. to 10° C. For fabrication of the PCL framework, pneumatic pressure is applied in the range from 400 to 650 kPa. The composition in the form of pre-gel is dispensed by using a plunger-based low-dosage dispensing system. In addition, the printing may be also carried out by dispensing only the composition in the form of pre-gel using a plunger-based low-dosage dispensing system, without using a polycaprolactone framework.

The crosslinking under UVA light may be carried out by irradiating UVA light having 315 to 400 nm of wavelength, preferably having about 360 nm of wavelength, for 1 to 10 minutes, preferably for about 3 minutes. By repeatedly performing said printing and said crosslinking under UVA light, i.e., the layer-by-layer process, a three-dimensional structure configuration becomes formed.

Step (ii) is carried out by performing thermal gelation of the three-dimensional structure configuration obtained from Step (i) at a temperature of 15° C. or more. The thermal gelation may be performed by standing the three-dimensional structure configuration at a humid incubator the temperature of which is maintained preferably at 20 to 40° C., more preferably at about 37° C., for 5 to 60 minutes, preferably for 20 to 30 minutes.

The present invention will be described in further detail with reference to the following examples. These examples are for illustrative purposes only and are not intended to limit the scope of the present invention.

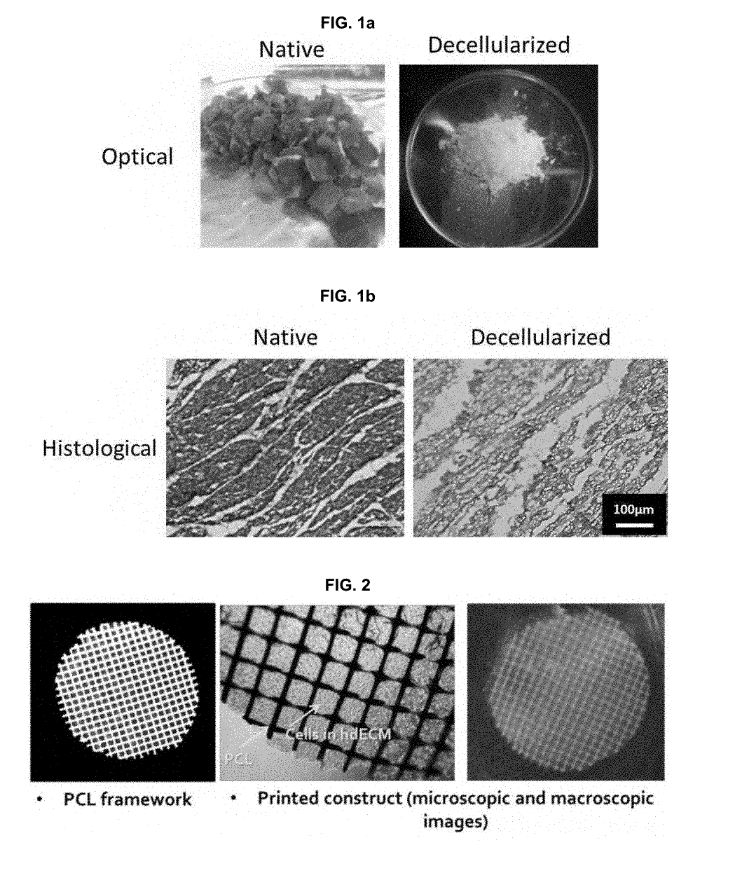

The decellularized extracellular matrix used in the following examples was obtained by using porcine heart tissues, according to the method disclosed in Falguni Pati, et al., Nat Commun. 5, 3935 (2014), and hereinafter referred to as ‘hdECM’. The obtained hdECM was finally lyophilized and stored in the freezer until the use thereof. The optical microscopic image and the histological image of the hdECM are as shown in FIGS. 1a and 1 b.

### Example 1: Preparation of the Three-Dimensional Printing Composition

Lyophilized hdECM was crushed into powder using a mortar and pestle with the help of liquid nitrogen. The hdECM powder (330 mg) was added to a solution of 0.5M acetic acid and then pepsin (33 mg) (P7125, Sigma-Aldrich) was added thereto. The mixture was stirred at room temperature for 48 h. While maintaining the temperature of the resulting solution at 10° C. or less, riboflavin (2 mg) was added thereto. The pH of the resulting solution was adjusted to about pH 7 with dropwise addition of cold (10° C. or less) 10M NaOH solution. The obtained solution in the form of pre-gel was stored in the refrigerator at about 4° C.

### Example 2: Preparation of the Three-Dimensional Structure

A three-dimensional structure was fabricated using the three-dimensional printing composition obtained in Example 1, according to the method disclosed in Falguni Pati, et al., Nat Commun. 5, 3935 (2014). Specifically, the polycaprolactone (PCL) framework was loaded into the syringe (first syringe) of the multi-head tissue/organ printing system (Jin-Hyung Shim et al., J. Micromech. Microeng. 22 085014 (2012)) and then heated to about 80° C. to melt the polymer. The three-dimensional printing composition in the form of pre-gel obtained in Example 1 was loaded to the other syringe (second syringe) and then maintained at temperatures below about 10° C. Pneumatic pressure of about 600 kPa was applied to the first syringe to fabricate the thin PCL framework of 120 μm thickness having a line width of less than about 100 μm, with a gap of about 300 μm. The contents in the second syringe was dispensed over the PCL framework and then the UVA light of about 360 nm was irradiated thereon for 3 minutes to crosslink the composition.

Then, the dispensing processes of the contents in the second syringe and then the layer-by-layer processes through the crosslinking were repeatedly carried out to form a three-dimensional structure configuration. The resulting three-dimensional structure configuration was placed in a humid incubator (the temperature thereof: about 37° C.) and then subject to thermal gelation by standing for 30 minutes to prepare a three-dimensional structure. The resulting three-dimensional structure has a thickness of about 300 to 400 μm. An example of the configuration is as shown in FIG. 2.

### Example 3: Preparation of the Three-Dimensional Structure

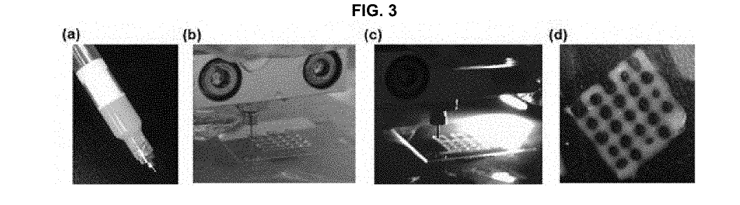

A three-dimensional structure was fabricated according to the same procedures as in Example 2, except that the PCL framework was not used. That is, the three-dimensional printing composition in the form of pre-gel obtained in Example 1 was loaded to the syringe of the multi-head tissue/organ printing system (Jin-Hyung Shim et al., J. Micromech. Microeng. 22 085014 (2012)) and then maintained at temperatures below about 10° C. Pneumatic pressure of about 600 kPa was applied to the syringe so as to dispense the contents therein and then the UVA light of about 360 nm was irradiated thereon for 3 minutes to crosslink the composition. Then, the dispensing processes of the contents in the second syringe and then the layer-by-layer processes through the crosslinking were repeatedly carried out to form a three-dimensional structure configuration. The resulting three-dimensional structure configuration was placed in a humid incubator (the temperature thereof: about 37° C.) and then subject to thermal gelation by standing for 30 minutes to prepare a three-dimensional structure. The resulting three-dimensional structure has a thickness of about 400 μm. An example of the configuration is as shown in FIG. 3.

### Comparative Example

The solution in the form of pre-gel was prepared according to the same procedures as in Example 1, except that riboflavin was not used.

### Experimental Example

The solution in the form of pre-gel obtained in Example 1 was subject to crosslinking by irradiating the UVA light of about 360 nm thereon for 3 minutes, placed in a humid incubator (the temperature thereof: about 37° C.), and then subject to thermal gelation by standing for 30 minutes to form a hydrogel (Hydrogel A). And also, the solution in the form of pre-gel obtained in Comparative Example was placed in a humid incubator (the temperature thereof: about 37° C.) and then subject to thermal gelation by standing for 30 minutes to form a hydrogel (Hydrogel B). The complex modulus at frequency of 1 rad/s was measured for each of the obtained hydrogels, and the results are shown in Table 1 below.

TABLE 1

| | Modulus (n = 3, 1 rad/s) |
|---|---|
| Hydrogel A | 10.58 ± 3.4 kPa |
| Hydrogel B | 0.33 ± 0.13 kPa |

As can be seen from the results in Table 1 above, the hydrogel obtained according to the present invention exhibits 10.58 kPa of modulus at frequency of 1 rad/s, which shows at least about 30-fold improvement in strength by the crosslinking.

1. A three-dimensional printing composition comprising a decellularized extracellular matrix; and riboflavin as a crosslinking agent.

2. The composition of claim 1, wherein the decellularized extracellular matrix is obtained by decellularization of heart tissues, cartilage tissues, bone tissues, adipose tissues, muscle tissues, skin tissue, mucosal epithelial tissues, amnion tissues, or corneal tissues which are externally discharged from the body.

3. The composition of claim 1, wherein the decellularized extracellular matrix is present in an amount ranging from 1 to 4% by weight, based on the total weight of the composition.

4. The composition of claim 1, wherein the riboflavin is present in an amount ranging from 0.001 to 0.1% by weight, based on the total weight of the composition.

5. The composition of claim 1, further comprising one or more acids selected from the group consisting of acetic acid and hydrochloric acid; one or more proteinases selected from the group consisting of pepsin and matrix metalloproteinase; and a pH adjusting agent.

6. The composition of claim 5, comprising 1 to 4% by weight of the decellularized extracellular matrix; 0.001 to 0.1% by weight of the riboflavin; 0.03 to 30% by weight of one or more acids selected from the group consisting of acetic acid and hydrochloric acid; 0.1 to 0.4% by weight of one or more proteinases selected from the group consisting of pepsin and matrix metalloproteinase; and a pH adjusting agent, based on the total weight of the composition.

7. The composition of claim 5, wherein the viscosity at 1 s⁻¹ shear rate when measured at 15° C. ranges from 1 to 30 Pa·S.

8. A method for preparing a three-dimensional printing composition comprising:
(a) adding a decellularized extracellular matrix to one or more acid solutions selected from the group consisting of acetic acid and hydrochloric acid,
(b) adding one or more proteinases selected from the group consisting of pepsin and matrix metalloproteinase to the solution obtained from Step (a), followed by stirring the mixture to obtain a solution, and
(c) adding riboflavin and a pH adjusting agent to the solution obtained from Step (b).

9. The method of claim 8, wherein the decellularized extracellular matrix is obtained by decellularization of heart tissues, cartilage tissues, bone tissues, adipose tissues, muscle tissues, skin tissue, mucosal epithelial tissues, amnion tissues, or corneal tissues which are externally discharged from the body.

10. The method of claim 8, wherein the decellularized extracellular matrix is used in an amount ranging from 1 to 4% by weight, based on the total weight of the composition.

11. The method of claim 8, wherein the riboflavin is used in an amount ranging from 0.001 to 0.1% by weight, based on the total weight of the composition.

12. A method for preparing a three-dimensional structure comprising:
(i) performing a printing process using the three-dimensional printing composition of claim 1, and a layer-by-layer process through crosslinking under UVA light, to form a three-dimensional structure configuration; and
(ii) performing thermal gelation of the three-dimensional structure configuration obtained from Step (i) at a temperature of 15° C. or more, to prepare a three-dimensional structure.

13. The method of claim 12, wherein the crosslinking in each layer-by-layer process is performed for 1 to 10 minutes.
